(19) United States
(12) Patent Application Publication
(10) Pub. No.: US 2026/0053860 A1
(43) Pub. Date: Feb. 26, 2026

(54) METHOD OF PROMOTING RETURN OF ESTRUS IN ANIMAL AND AGENT FOR PROMOTING RETURN TO ESTRUS IN ANIMAL

(71) Applicant: Anicom Holdings, Inc., Tokyo (JP)

(72) Inventors: Ryuta KUWANA, Tokyo (JP); Ryo HORIE, Tokyo (JP); Hisashi MATSUMOTO, Tokyo (JP); Yuri ISHIWATA, Tokyo (JP); Kanako WASAKI, Tokyo (JP); Shiori SATO, Tokyo (JP); Akane KIDA, Tokyo (JP)

(73) Assignee: Anicom Holdings, Inc., Tokyo (JP)

(21) Appl. No.: 19/252,395

(22) Filed: Jun. 27, 2025

(30) Foreign Application Priority Data
Jun. 28, 2024 (JP) 2024-105355

(51) Int. Cl. A61K 35/28; A61P 15/08

(52) U.S. Cl. CPC A61K 35/28; A61P 15/08

(57) ABSTRACT

A method of promoting return to estrus in an animal and an agent for promoting return to estrus in an animal are provided. The method is a method of promoting return to estrus in an animal, characterized by including a step of administering mesenchymal stromal cells or a composition containing mesenchymal stromal cells to the animal. The mesenchymal stromal cells are preferably adipose-derived stem cells, and the animal is preferably a farm animal, a dog, or a cat.

### CROSS-REFERENCE TO RELATED APPLICATION

This non-provisional application claims the benefit under 35 U.S.C. § 119(a) to Patent Application No. 2024-105355, filed in Japan on Jun. 28, 2024, which is hereby expressly incorporated by reference into the present application.

### FIELD OF THE INVENTION

The present invention relates to a method of promoting return to estrus in an animal and an agent for promoting return to estrus in an animal.

### BACKGROUND OF THE INVENTION

For farms and breeders that handle farm animals and pets, improving the efficiency of animal reproduction is a crucial issue from a management perspective. Measures to improve reproductive efficiency include enhancing the health of both offspring and parents, reducing stillbirths and postpartum deaths, and shortening the childbirth interval.

Meanwhile, in recent years, the concept of animal welfare has gained widespread recognition throughout the world. The World Organisation for Animal Health (WOAH), an international organization that aims to improve animal health worldwide, defines animal welfare as the “physical and mental state of animals concerning the conditions in which they live and die.” The Ministry of Agriculture, Forestry and Fisheries in Japan believes that raising farm animals in a comfortable environment is crucial to reducing stress and illness in farm animals, ultimately leading to improved productivity and the production of safe livestock products. The ministry is working to popularize farm animal rearing management based on the concept of animal welfare.

Given the above background, there is a need to improve reproductive efficiency while placing as little strain as possible on animals. As part of research related to improving reproductive efficiency, studies have been conducted to reduce the number of days required for return to estrus, that is, to quickly restore animals to a state in which they can reproduce during the postpartum period.

For example, Non-Patent Document 1 discloses a study of factors affecting the return to estrus during the postpartum period, particularly the relationship between feeding and blood components.

Furthermore, Non-Patent Document 2 investigated the effectiveness of supplementing energy intake by feeding medium-chain triglycerides (MCT) and administering gonadotropic hormones as countermeasures to the delay in return to estrus in breeding sows during the summer, and reported that MCT was not effective in reducing the number of days required for return to estrus, but the administration of hormones was effective.

However, there is a limit on avoiding a delay in return to estrus by changing or improving feed. Although the administration of hormones is expected to be effective, there are some countries in which the administration of hormones is prohibited. For these reasons, there is still room for improvement in methods for reducing the number of days (or hours) required for return to estrus.

Non-Patent Document 1: The Tohoku journal of veterinary clinics, vol. 16, No. 1

Non-Patent Document 2: Research bulletin of the Aichi-ken Agricultural Research Center, 47:151-154(2015)

### SUMMARY OF THE INVENTION

As described above, a novel method of reducing the number of days required for return to estrus in an animal is required. The disclosed exemplary embodiments provide a method of promoting return to estrus in an animal and an agent for promoting the return to estrus in an animal.

As a result of intensive studies aimed at solving the above problems, the present inventors found that administering mesenchymal stromal cells to animals makes it possible to promote the return to estrus. This has led to the completion of the present invention.

Specifically, the present invention relates to the following [1] to [9].
[1] A method of promoting return to estrus in an animal, characterized by including a step of administering mesenchymal stromal cells or a composition containing mesenchymal stromal cells to the animal.
[2] The method of promoting return to estrus in an animal according to [1], wherein the mesenchymal stromal cells are adipose-derived stem cells.
[3] The method of promoting return to estrus in an animal according to [1], wherein the animal is a farm animal, a dog, or a cat.
[4] The method of promoting return to estrus in an animal according to [3], wherein the farm animal is a bovine, a horse, or a pig.
[5] The method of promoting return to estrus in an animal according to any one of [1] to [4], wherein the animal is a postpartum animal.
[6] The method of promoting return to estrus in an animal according to [1], whereby the number of days required for return to estrus can be reduced.
[7] A method of promoting recovery of reproductive function in an animal, characterized by including a step of administering mesenchymal stromal cells or a composition containing mesenchymal stromal cells to the animal after delivery.
[8] An agent for promoting return to estrus, characterized by containing animal mesenchymal stromal cells.
[9] An agent for promoting recovery of reproductive function in an animal, characterized by containing animal mesenchymal stromal cells.

According to the present invention, it becomes possible to provide a method of promoting return to estrus in an animal and an agent for promoting the return to estrus in an animal.

### BRIEF DESCRIPTION OF THE DRAWINGS

FIG. 1A is a graph showing the results of Examples determined by actual measurements plus breeder interviews (the average of the actual measurements and the values obtained by breeder interviews), with the average number of days required for return to estrus. FIG. 1B is a graph showing the results of Examples with the distribution of the number of days required for return to estrus.

FIG. 2A is a graph showing the results of Examples and showing average value for all individuals of the actual number of days required for return to estrus. FIG. 2B shows the distribution value for all individuals of the actual number of days required for return to estrus.

### DETAILED DESCRIPTION

### Method of Promoting Return to Estrus in Animal

The method of promoting return to estrus in an animal of the present invention is characterized by including a step of administering mesenchymal stromal cells or a composition containing mesenchymal stromal cells to the animal.

In the present invention, promoting return to estrus in an animal means promoting resumption of estrus in an animal, preferably hastening return to estrus in an animal, and more preferably reducing the number of days required for return to estrus in an animal. Return to estrus refers to the resumption of estrus in a female animal whose estrus has temporarily stopped for various reasons, such as parturition, miscarriage, or ovarian quiescence. Whether estrus has begun can be determined by employing generally known standards for estrus in each animal, for example, by a breeder visually inspecting the animal. By promoting the return to estrus, the time during which an animal becomes ready to conceive after delivery can be reduced. In addition, by promoting the return to estrus, it is possible to suppress occurrence of delay in return to estrus. In other words, the method of promoting return to estrus in an animal of the present invention includes, preferably, a method of suppressing occurrence of delay in return to estrus in an animal.

### Target for Administration

In the present invention, there are no particular limitations on animals (excluding humans) to which mesenchymal stromal cells or a composition containing mesenchymal stromal cells is administered. Preferably, the animal is a female animal. Examples of suitable animals include farm animals, dogs, and cats, with farm animals being preferably bovines, horses, pigs, goats, and sheep.

There are no particular limitations on the age of a target animal for administration, and the animal is preferably of an age at which mesenchymal stromal cells can be administered or at an age at which pregnancy and childbirth are possible.

### Mesenchymal Stromal Cells

Mesenchymal stromal cells (MSCs) are somatic stem cells present in the mammalian body. Since mesenchymal stromal cells can be easily cultured outside the body, thereby amplifying their number, and they exhibit angiogenic, immunosuppressive, and anti-inflammatory effects, clinical studies and trials are being conducted on them as immunosuppressants and therapeutic agents for intractable autoimmune diseases. They are attracting attention as a key field in cell therapy.

Mesenchymal stromal cells are a type of adult stem cell that have been reported to be found in various tissues, including bone marrow, fat, umbilical cord, and synovium (the tissue surrounding joints). The mesenchymal stromal cells used in the present invention preferably have the following characteristics: (1) they are positive for the cell surface antigens CD44, CD73, CD90, and CD105 are positive, and CD14, CD19, CD34, CD45, and MHC Class II are negative; (2) they adhere to plastic and grow with a fibroblast-like morphology under standard culture conditions; and (3) they are capable of differentiating into adipocytes, osteoblasts, and chondroblasts.

The above description of the cell surface antigens in (1) is one preferred example, and known surface antigens can be selected for each animal species. For example, in dogs, MSCs are preferably positive for CD44 and CD90, and negative for CD45 and MHC class II. Furthermore, MSCs are more preferably positive for any one or more of CD29, CD73, and CD105. In cats, MSCs are preferably positive for CD29 and CD44, and negative for CD34 and MHC class II. Furthermore, MSCs are preferably positive for any one or more of CD73, CD90, and CD166. In bovines, MSCs are preferably positive for any one or more of CD29, CD44, CD73, CD90, CD105 and CD166, and negative for any one or more of CD11b, CD14, CD31, CD34, CD45 and CD117. In horses, MSCs are preferably positive for one or more of CD29, CD44, CD73, CD90, CD105 and CD166, and negative for one or more of CD11b, CD14, CD31, CD34, CD45 and MHC class II. In goats, MSCs are preferably positive for any one or more of CD29, CD44, CD73, CD90 and CD105, and negative for any one or more of CD11b, CD14, CD31, CD34 and CD45. The above examples can be modified as appropriate, and the surface antigens can be modified and selected according to publicly known or commonly known information.

Mesenchymal stromal cells can be harvested from bone marrow, but also from adipose tissue. Subcutaneous fat has the following advantages over bone marrow: (1) tissue sampling is relatively easy; and (2) the amount of mesenchymal stromal cells contained per unit volume of tissue is several hundred times higher than that of bone marrow.

Subcutaneous fat is a preferred resource for mesenchymal stromal cells because it is easy to secure the required number of cells. Therefore, in the present invention, it is preferable to use adipose-derived stem cells as mesenchymal stromal cells. The adipose-derived stem cells may contain cells other than mesenchymal stromal cells.

### Adipose-Derived Stem Cells

Adipose-derived stem cells (ASCs) are somatic stem cells contained in adipose tissue. For example, they can be harvested from subcutaneous adipose tissue. In the present invention, adipose-derived stem cells from animals, particularly mammals, are preferably used. Either autologous or allogeneic stem cells may be used; however, allogeneic adipose-derived stem cells are preferred, considering the cost and effort involved in administering them regularly. In the case of using autologous adipose-derived stem cells, stem cells are isolated from the adipose tissue of the target animal for administration. These stem cells are then cultured and administered back to the animal. In the case of using allogeneic adipose-derived stem cells, adipose-derived stem cells prepared in advance can be used. In the case of allogeneic stem cells, mesenchymal stromal cells or adipose-derived stem cells derived from an animal of the same species as the target animal for administration are preferred.

Adipose-derived stem cells can be prepared by harvesting and culturing them using known methods. For example, in the case of allogeneic adipose-derived stem cells, subcutaneous fat is collected from healthy animals under anesthesia using surgical instruments, and the wound is closed by suturing. The collecting site is the abdomen. Thereafter, adipose-derived stem cells are collected from the obtained subcutaneous fat by enzyme treatment, cultured for several days, for example, about seven days, and then cryopreserved, thereby yielding adipose-derived stem cells. The solution is thawed before use, if appropriate.

For adipose-derived stem cells, surface antigen markers can include those similar to the surface antigens of the mesenchymal stromal cells described above. For dogs, adipose-derived stem cells are preferably those with CD90≥85% and/or CD44≥85%. They are also preferably with CD45≤3% and MHCII≤3%. For cats, adipose-derived stem cells are preferably those with CD29≥50% and/or CD44≥80%. They are also preferably with CD34≤3% and MHCII≤3%. The term “%” used herein refers to the percentage of cells contained in a cell population that are positive for a particular marker, and can be measured by known methods. In a case in which these markers are confirmed, it is not necessary to also confirm the preferred mesenchymal stromal cell markers described above.

In a case in which allogeneic adipose-derived stem cells are administered, an immunosuppressant may be used in combination; however, it is also possible to administer them without concomitant use of an immunosuppressant. In a case in which no immunosuppressant is used concomitantly, it is preferable to check for the presence or absence of a marker that affects the immune response, such as MHC class II. In a specific example, when the MHC class II positivity rate of the adipose-derived stem cells to be administered exceeds 3%, there is a high possibility that they will cause an immune rejection reaction. Therefore, it is preferable to avoid administering them to animals.

### Preparation of Adipose-Derived Stem Cells

An example of a procedure for preparing adipose-derived stem cells (procedures such as culture, selection, collection, freezing, and thawing) will be described below. Specifically, one embodiment of a step for isolating and obtaining adipose-derived stem cells from adipose tissue contained in dogs, as an example of an animal, is described. However, the present invention is not to be construed as being limited thereto. Similar procedures can also be used for animals other than dogs, and may be modified as appropriate depending on the animal species.

### (1) Preparation of Cell Population from Adipose Tissue

The adipose tissue is collected by a procedure such as resection under anesthesia. The collected adipose tissue is exposed to 70% ethanol for a short period, thereby cleaning and sterilizing bacteria, viruses, and other microorganisms that may be adhering to the tissue. It is then immersed in a buffer solution or culture medium and subjected to the following enzyme treatment. The sterilization treatment may include a method using a known sterilizing agent, such as a 10% iodine solution.

In the enzymatic treatment, the adipose tissue is enzymatically digested with an enzyme solution of collagenase Type I (from 0.1 to 5 mg/mL) at 37° C. for from 30 to 120 minutes, thereby obtaining a cell population (solution) containing adipose-derived stem cells. Examples of enzymes used for decomposing the adipose tissue include trypsin, dispase, and other commercially available digestive enzymes for adipose tissue, which are used in known methods.

To fractionate the cell population including the adipose-derived stem cells, centrifugation is performed. An enzyme-treated solution is dispensed into a 50-mL centrifuge tube and centrifuged at from 750 to 1500 G (1.0 G=9.80665 m/s²). A cell population including adipose-derived stem cells is then collected from the precipitated fraction at the bottom of the tube. The centrifugal acceleration may be varied depending on the amount of adipose tissue. The precipitated fraction is added to a new centrifuge tube containing D-PBS and washed, thereby removing remaining oil components and other impurities. The solution containing the adipose-derived stem cells is filtered through a cell strainer having a pore size of from 70 to 100 μm, thereby removing extracellular matrix (ECM), undecomposed adipose tissue, undegraded adipose tissue, and the like. The buffer solution, filtration filter, and equipment used as described in this section may also include other known buffer solutions, filtration membranes, and equipment to be used.

### (2) Selective Culture of Adherent Fibroblast-Like Cells and Collection of Cells (P0)

The solution containing the cells mentioned above is centrifuged at from 750 to 1500 G, and the precipitated fraction at the bottom of the tube is collected as a cell population including adipose-derived stem cells. After adding an appropriate medium thereto to suspend the cells, the suspension is transferred to a 225 cm² culture flask and cultured for from 7 to 10 days, with the medium being repeatedly washed and changed every 3 to 4 days. The culture environment in the culture incubator is maintained at 37° C. and the carbon dioxide concentration is 5%. As the culture medium, a medium for ordinary animal cell culture can be used. For example, this includes the use of Dulbecco's Modified Eagle's Medium (DMEM) (e.g., FUJIFILM Wako Pure Chemical Corporation), α-MEM (e.g., FUJIFILM Wako Pure Chemical Corporation), DMEM:Ham's F12 mixed medium (1:1) (e.g., FUJIFILM Wako Pure Chemical Corporation), and Ham's F12 Medium (e.g., FUJIFILM Wako Pure Chemical Corporation). The serum to be added to the medium may be derived from fetal bovine serum (FBS), human serum, sheep serum, or the like. The amount of serum or serum substitute added may be within a range of, for example, from 5% (v/v) to 30% (v/v) during culture.

To collect the proliferated cells, the collection procedures follow an ordinary method for detaching cells adhered to the bottom of a flask. For example, the cells can be easily collected by detaching them after enzyme treatment (such as trypsin or dispase treatment). The detached cells are suspended in a medium containing fetal bovine serum so as to inhibit trypsin activity, and then centrifuged at from 500 to 1500 G, thereby removing medium components. The cell population is then washed again with D-PBS and used as a passage 0 (P0) adipose-derived stem cell suspension.

### (3) Selective Culture of Adipose-Derived Stem Cells and Collection of Cells (P1)

P0 adipose-derived stem cells are prepared at a concentration of from 3 to 5×10⁴ cells/cm². After adding an appropriate medium to suspend the cells, the suspension is transferred to a 225-cm² culture flask and cultured for from 7 to 10 days, with the medium being repeatedly washed and changed every 3 to 4 days. The culture environment in the culture incubator is maintained at 37° C. and the carbon dioxide concentration is 5%. As the culture medium, a standard culture medium for animal cells can be used, as in the case of culturing P0 cells.

To collect the proliferated cells (P1), the collection procedures follow an ordinary method for detaching cells adhered to the bottom of a flask. For example, the cells can be easily collected by detaching them after enzyme treatment (such as trypsin or dispase treatment). The detached cells are suspended in a medium containing FBS so as to inhibit trypsin activity, washed with D-PBS, and then centrifuged at from 500 to 1500 G, thereby washing off the contained medium components. The cell population is then washed again with D-PBS and then yielded as a mesenchymal stromal cell suspension for Passage 1 (P1).

### (4) Cell Cryopreservation Method

The P0 cells and P1 cells are suspended in a cryopreservation solution at a concentration of from 1×10⁶ to 1×10⁷ cells/mL, according to standard cell freezing methods and dispensed into cryotubes, followed by slow freezing in a −80° C. freezer using a slow freezing device. A standard preservation solution for freezing animal cells can be used as a cryopreservation solution. This also includes the use of commercially available cell freezing solutions containing/not containing dimethyl sulfoxide, such as CELL BANKER I (TAKARA), COS BANKER (Cosmo Bio Co., Ltd.), and Banbanker (NIPPON Genetics Co., Ltd.), or the like. The dimethyl sulfoxide (DMSO) contained in the freezing solution ranges from 0% (v/v) to 10% (v/v), for example.

### (5) Cell Thawing Method

The frozen P0 and P1 cells are thawed according to an ordinary cell thawing method. The thawed cell suspension is centrifuged at from 500 to 1500 G so as to remove the cell freezing solution, after which a buffer solution such as D-PBS is added to the cell population, and washing and centrifugation procedures are repeated. Examples of a method of thawing a frozen tube include rapid thawing using an automatic thawing device, such as a cell freezing and thawing station Thawstar (BioCision).

### Composition Containing Mesenchymal Stromal Cells

The composition containing mesenchymal stromal cells may also contain, in addition to the mesenchymal stromal cells, a solvent, auxiliary components, and the like. Preferably it is a cell suspension. The preferred composition of the mesenchymal stromal cell-containing composition is the same as that for the agent for promoting return to estrus, which will be described later.

### Administration Method

The method of administering the mesenchymal stromal cells is not particularly limited, and any known method can be used. From the viewpoint of reducing the burden on the animal, intravenous drip infusion, intravenous injection, subcutaneous injection, or intramuscular injection is preferred. Subcutaneous or intramuscular injection is preferred, as it allows for a shorter administration time compared to intravenous drip infusion.

### Dosage

The preferred dosage of mesenchymal stromal cells per administration is from 1×10⁴ cells/kg (body weight) to 1×10⁸ cells/kg (body weight), and more preferably from 1×10⁵ cells/kg to 1×10⁷ cells/kg (body weight).

### Administration Schedule

In the method of the present invention, mesenchymal stromal cells are administered one or more times. For example, administering mesenchymal stromal cells once, either before pregnancy or after delivery, is expected to have a long-term effect in promoting return to estrus. Periodic administration is also preferred. Regular administration is expected to promote continued promoting effect in return to estrus thereafter. For example, administration schedules include administration once a year or once every few months. The timing of administration is preferably after birth, and more preferably once after birth. Preferably, the administration is also performed once for each birth.

### Method of Promoting Recovery of Reproductive Function

The method of promoting recovery of reproductive function in an animal of the present invention is characterized by including a step of administering mesenchymal stromal cells or a composition containing mesenchymal stromal cells to the animal.

In the present invention, the method of promoting recovery of reproductive function in an animal refers to promoting the recovery of reproductive function such that the animal becomes capable of breeding again after delivery, and preferably includes the recovery of physical strength and the promotion of the mother's health after delivery.

The mesenchymal stromal cells, the target for administration, the administration method, and the like are the same as those in the above-described method of promoting return to estrus.

### Agent for Promoting Return to Estrus

The agent for promoting return to estrus of the present invention is characterized by containing mesenchymal stromal cells and used for promoting return to estrus in an animal. In other words, the agent for promoting return to estrus of the present invention is an agent or composition for use in promoting return to estrus in an animal.

The agent for promoting return to estrus of the present invention contains, as an active ingredient, mesenchymal stromal cells, and preferably adipose-derived stem cells. Preferably it is a cell suspension. The concentration of mesenchymal stromal cells in the agent for promoting return to estrus is preferably, it is 1×10⁴ cells/mL or more, more preferably, 1×10⁵ cells/mL or more, and still more preferably, 4×10⁶ cells/mL or more. The agent for promoting return to estrus of the present invention may contain, in addition to the active ingredient, auxiliary components, including a solvent such as water and a buffer solution.

Examples of the buffer solution include a Ringer's solution, an L-sodium lactate Ringer's solution, a 5% glucose lactate Ringer's solution, an acetate Ringer's solution, a 5% glucose acetate Ringer's solution, a Dulbecco's phosphate buffered saline (D-PBS), saline, Good's buffer, a Hank's balanced salt solution, phosphate buffer (PBS), imidazole buffer, triethanolamine hydrochloride buffer (TEA), or a combination thereof.

The agent for promoting return to estrus of the present invention may further contain a nutrient and a chelating agent. In addition, the agent for promoting return to estrus of the present invention preferably does not substantially contain any medium components for animal cells, an organic solvent, and dextran.

The preferable agent for promoting return to estrus of the present invention contains animal mesenchymal stromal cells, and preferably adipose-derived stem cells, suspended in a buffer solution, the concentration of mesenchymal stromal cells in the agent for promoting return to estrus is 1×10⁵ cells/mL or more, and the buffer solution preferably contains at least one of ascorbic acid, ethylenediaminetetraacetic acid, and citric acid, does not contain any sugars other than trehalose, and does not contain 2% or more of trehalose. For the agent for promoting return to estrus of the present invention, the concentration of ascorbic acid in the buffer solution is preferably from 1 mmol/L to 150 mmol/L, and the concentration of ethylenediaminetetraacetic acid or citric acid in the buffer solution is preferably from 1 mmol/L to 100 mmol/L.

The agent for promoting return to estrus of the present invention may be stored frozen or kept at from 0° C. to 10° C.

### Dosage Form

The dosage form of the agent for promoting return to estrus of the present invention is not particularly limited, and may be any known dosage form. For example, examples of the dosage form include cell suspensions, injections, drip infusions, and capsules. The dosage form is preferably a cell suspension in which cells are suspended in a solvent, and more preferably an injection or drip infusion solution consisting of the cell suspension.

The target for administration of the agent for promoting return to estrus of the present invention is the same as that in the above-described method of promoting return to estrus.

### Agent for Promoting Recovery of Reproductive Function

The agent for promoting recovery of reproductive function of the present invention is characterized by containing mesenchymal stromal cells and is used for promoting recovery of reproductive function in an animal. In other words, the agent for promoting recovery of reproductive function of the present invention is an agent or composition for used in promoting recovery of reproductive function in an animal after birth. The components, target for administration, and the like are the same as those of the above-described agent for promoting return to estrus.

### EXAMPLES

The abbreviations and trade names in Examples have the following meanings.
PBS: D-PBS(−) (FUJIFILM Wako Pure Chemical Corporation)
Sodium ascorbate: L(+)-sodium ascorbate (FUJIFILM Wako Pure Chemical Corporation)

### Preparation of Dog Adipose-Derived Stem Cells

### (1) Preparation of Cell Population from Adipose Tissue

Adipose tissue was obtained from subcutaneous fat of healthy dogs. The collected adipose tissue was sterilized briefly with 70% ethanol and then enzymatically digested with an enzyme solution of collagenase Type I (from 0.1 to 5 mg/mL) at 37° C. for 60 minutes, thereby obtaining a cell population (solution) containing adipose-derived stem cells.

To fractionate the cell population including adipose-derived stem cells, the above-described enzyme-treated solution was dispensed into 50-mL centrifuge tubes and centrifuged at from 750 to 1500 g. The precipitated fraction at the bottom of the tube was then collected as the cell population. The precipitate fraction was transferred to a new centrifuge tube containing PBS and rinsed, thereby removing any remaining fat and oil components, as well as other impurities. Subsequently, the extracellular matrix and undecomposed adipose tissue in the adipose tissue were removed by filtration through a cell strainer having a pore size of 70 μm to prepare a solution containing cells.

### (2) Culture and Collection of Adipose-Derived Stem Cells

The solution containing the cells mentioned above was centrifuged at 1500 G, and the precipitated fraction at the bottom of the tube was collected as a cell population including adipose-derived stem cells. After adding an appropriate medium thereto to suspend the cells, the suspension was transferred to a 225 cm² culture flask and cultured for from 7 to 10 days, with the medium being repeatedly washed and changed every 3 to 4 days. The culture was carried out at 37° C. and at a carbon dioxide concentration of 5%. The culture medium used was Mesenchymal Stem Cell Growth Medium Bullet Kit (trademark) (MSCGM, Lonza K.K.).

To collect the proliferated cells, the collection procedures followed an ordinary method for detaching cells adhered to the bottom of a flask. The cells were collected by reaction with 0.05% (v/v) trypsin at 37° C. for 5 minutes. The detached cells were suspended in a medium containing fetal bovine serum (FBS, FUJIFILM Wako Pure Chemical Corporation) and then centrifuged at from 500 to 1500 G, thereby removing trypsin. The cells were then suspended in the medium, thereby obtaining a P0 adipose-derived stem cell suspension.

### (3) Culture and Collection of Adipose-Derived Stem Cells

The suspension of P0 mesenchymal stromal cells was transferred to a 225 cm² culture flask and cultured for from 7 to 10 days, with the medium being repeatedly washed and changed every 3 to 4 days. The culture was carried out at 37° C. and at a carbon dioxide concentration of 5%. The culture medium used was D-MEM (FUJIFILM Wako Pure Chemical Corporation) or the like supplemented with FBS. Similar to the collection of P0, the cells were reacted with trypsin and then centrifuged, thereby obtaining a P1 adipose-derived stem cell suspension.

### (4) Cell Cryopreservation

The P1 cells were suspended in a cryopreservation solution at a concentration of from 1×10⁶ to 5×10⁶ cells/mL, according to standard cell freezing methods and dispensed into cryotubes, followed by slow freezing in a −80° C. freezer using a slow freezing device. The cryopreservation solution used was a commercially available cell freezing solution containing dimethyl sulfoxide (DMSO). They were then stored in an ultralow temperature freezer (from −80° C. to −150° C.) or in liquid nitrogen (−196° C.).

### (5) Cell Thawing

The frozen P1 cells were thawed using an automatic thawing device, a cell freezing and thawing station Thawstar (BioCision), according to a standard cell thawing method. The mixture was centrifuged at 2000 G, thereby removing the cell freezing solution. The cell population was suspended in physiological saline and centrifuged, and then the cells were suspended in PBS containing 25 mM sodium ascorbate.

### Administration of Adipose-Derived Stem Cells to Dogs

The suspension of dog adipose-derived stem cells prepared as described above was administered once via intramuscular injection to 17 female dogs (including various breeds) immediately after giving birth (on average, 42.6 days after giving birth). The dosage was approximately 1×10⁶ cells/kg (body weight).

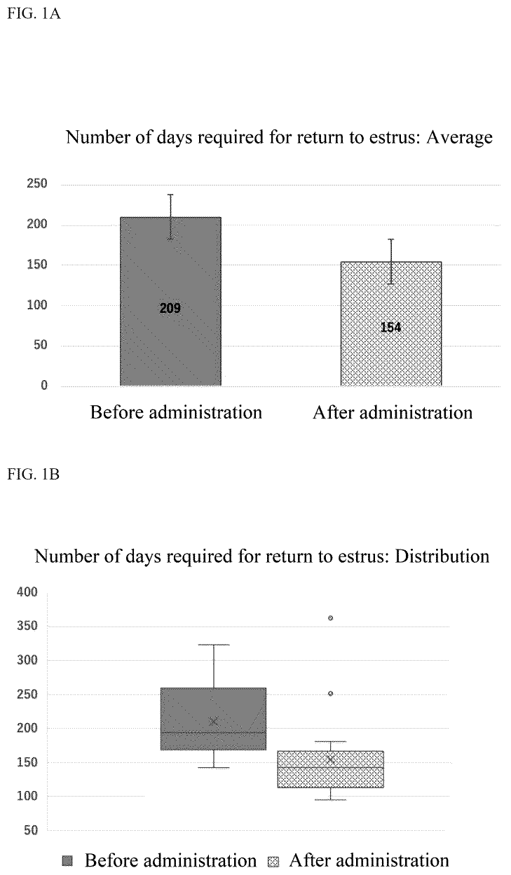

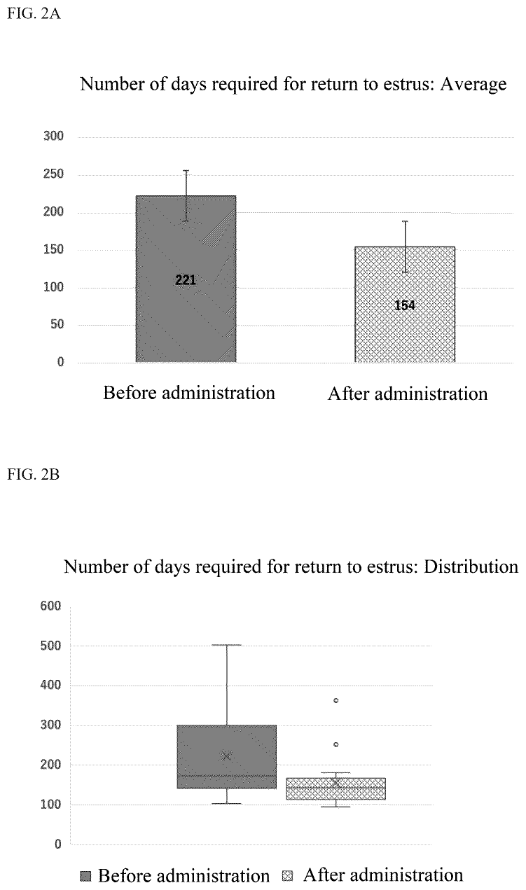

The number of days required for return to estrus was examined before and after administration. The number of days required for return to estrus was determined by counting the number of days from birth to the next estrus, and was confirmed by visual inspection (actual measurements) by the inventors or by checking the breeder's rearing records (breeder interview). The results are shown in FIGS. 1 and 2. FIG. 1 shows the number of days required for return to estrus as determined by actual measurements plus breeder interviews (the average of the actual measurements and the values obtained by breeder interviews), with (A) showing the average value for all individuals and (B) showing the distribution. FIG. 2 shows the actual number of days required for return to estrus, with (A) showing the average value for all individuals and (B) showing the distribution.

From FIGS. 1 and 2, it was found that the number of days required for return to estrus was shorter on average by 55 days after administration than before administration (actual measurements and results of breeder interviews; actual measurements were 67 days). Furthermore, the difference between the administration group and the non-administration group was confirmed to be significant by t-test. This demonstrates that administration of mesenchymal stromal cells can promote the return to estrus in animals. One possible reason for this effect is that damage to the mother's body after birth can be quickly recovered by the administration of mesenchymal stromal cells; however, this is not limited to the above. Based on such a speculation mechanism, it can be understood that similar effects would be achieved in animals other than dogs.

1. A method of promoting return to estrus in an animal, which comprises a step of administering mesenchymal stromal cells or a composition containing mesenchymal stromal cells to the animal.

2. The method of promoting return to estrus in an animal according to claim 1, wherein the mesenchymal stromal cells are adipose-derived stem cells.

3. The method of promoting return to estrus in an animal according to claim 1, wherein the animal is a farm animal, a dog, or a cat.

4. The method of promoting return to estrus in an animal according to claim 3, wherein the farm animal is a bovine, a horse, or a pig.

5. The method of promoting return to estrus in an animal according to claim 1, wherein the animal is a postpartum animal.

6. The method of promoting return to estrus in an animal according to claim 1, whereby the number of days required for return to estrus is reduced.

7. A method of promoting recovery of reproductive function in an animal, which comprises a step of administering mesenchymal stromal cells or a composition containing mesenchymal stromal cells to the animal after delivery.

8. An agent for promoting return to estrus, comprising animal mesenchymal stromal cells.

9. An agent for promoting recovery of reproductive function in an animal, comprising animal mesenchymal stromal cells.
